(19) United States
(12) Patent Application Publication
(10) Pub. No.: US 2013/0160195 A1
(43) Pub. Date: Jun. 27, 2013

(54) PORTABLE FAN AND BATTERY BOX FOR CLEARING FOG/PARTICLES IN GOGGLES OR FOR COOLING MASKS AND HELMETS

(71) Applicant: James Thomas Clement, Decatur, AL (US)

(72) Inventor: James Thomas Clement, Decatur, AL (US)

(21) Appl. No.: 13/726,517

(22) Filed: Dec. 24, 2012

Related U.S. Application Data
(60) Provisional application No. 61/579,583, filed on Dec. 22, 2011.

(51) Int. Cl. A61F 9/02

(52) U.S. Cl. CPC A61F 9/028
USPC 2/437

(57) ABSTRACT

A system for clearing fog and particles in goggles and for cooling masks and helmets includes a portable fan and battery box that can deliver a stream of air to a desired location. The stream of air can be provided on an inside face of eye-wear, for example, to prevent fogging. The stream of air can also be provided to an inside of a mask or helmet for cooling the user and providing increased comfort. The portable fan can be used with an air stream directing tube for directing the air stream to a desired location, or can be used as a fan for simply blowing a stream of air inside a mask or helmet.

### CROSS-REFERENCE TO RELATED APPLICATION

This application claims the benefit of priority of U.S. provisional application number 61/579,583, filed Dec. 22, 2011, the contents of which are herein incorporated by reference.

### BACKGROUND OF THE INVENTION

The present invention relates to goggle, helmet and mask accessories and, more particularly, to a portable fan and battery box for clearing fog and particles in goggles or for cooling masks, helmets and the like.

Goggles often fog up when worn, limiting the user's vision. Helmets and masks get hot, adding discomfort to the user.

Masks and helmets which already have a built in cooling system by the manufacturer tend to be very expensive. Moreover, the user is limited to the selection that includes these built in cooling systems.

Fog resistant goggles can be quite expensive as well and the user is again limited to the selection provided by the fog resistant goggle manufacturer. Moreover, the fog resistant properties and results achieved with fog resistant goggles can vary greatly.

As can be seen, there is a need for an apparatus to add a cooling system or anti-fogging system to goggles, helmets, masks and the like.

### SUMMARY OF THE INVENTION

In one aspect of the present invention, a safety device accessory comprises a fan unit operable to create an airflow; a battery unit operable to power the fan unit; and an attachment mechanism for removably attaching the fan unit and the battery unit to a user, wherein the airflow is directed to achieve at least one of 1) a first airflow into a helmet or mask of a user to provide user comfort, or 2) a second airflow onto a lens of eyewear of the user to prevent fogging thereof.

In another aspect of the present invention, a safety device accessory comprises a fan unit operable to create an airflow; a battery unit operable to power the fan unit; and an attachment point for removably attaching the fan unit and the battery unit to a helmet or mask work by a user, wherein the airflow is directed to achieve at least one of 1) a first airflow into the helmet or mask of a user to provide user comfort, or 2) a second airflow, delivered via an air outlet hose, onto a lens of eyewear of the user to prevent fogging thereof.

These and other features, aspects and advantages of the present invention will become better understood with reference to the following drawings, description and claims.

### BRIEF DESCRIPTION OF THE DRAWINGS

FIG. 1 is a perspective view of a user wearing a helmet and eye-wear having a portable fan and battery box formed therewith for cooling and preventing eye-wear fogging, according to an exemplary embodiment of the present invention;

FIG. 2 is a perspective view of the cooling system of FIG. 1;

FIG. 3 is a back perspective view of a helmet configured for eye-wear antifogging according to an exemplary embodiment of the present invention;

FIG. 4 is a front perspective view of the helmet with eye-wear antifogging function as shown in FIG. 3; and

FIG. 5 is a side view of the helmet with eye-wear antifogging function as shown in FIG. 3.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a system for clearing fog and particles in goggles and for cooling masks and helmets. The system includes a portable fan and battery box that can deliver a stream of air to a desired location. The stream of air can be provided on an inside face of eye-wear, for example, to prevent fogging. The stream of air can also be provided to an inside of a mask or helmet for cooling the user and providing increased comfort. The portable fan can be used with an air stream directing tube for directing the air stream to a desired location, or can be used as a fan for simply blowing a stream of air inside a mask or helmet. The system of the present invention has a greatly reduced cost as compared to fog resistant goggles or self-cooling masks and helmets. Moreover, the user can use virtually any goggle, helmet or mask and adapt it for use with the system of the present invention, without being limited to a particular design or style.

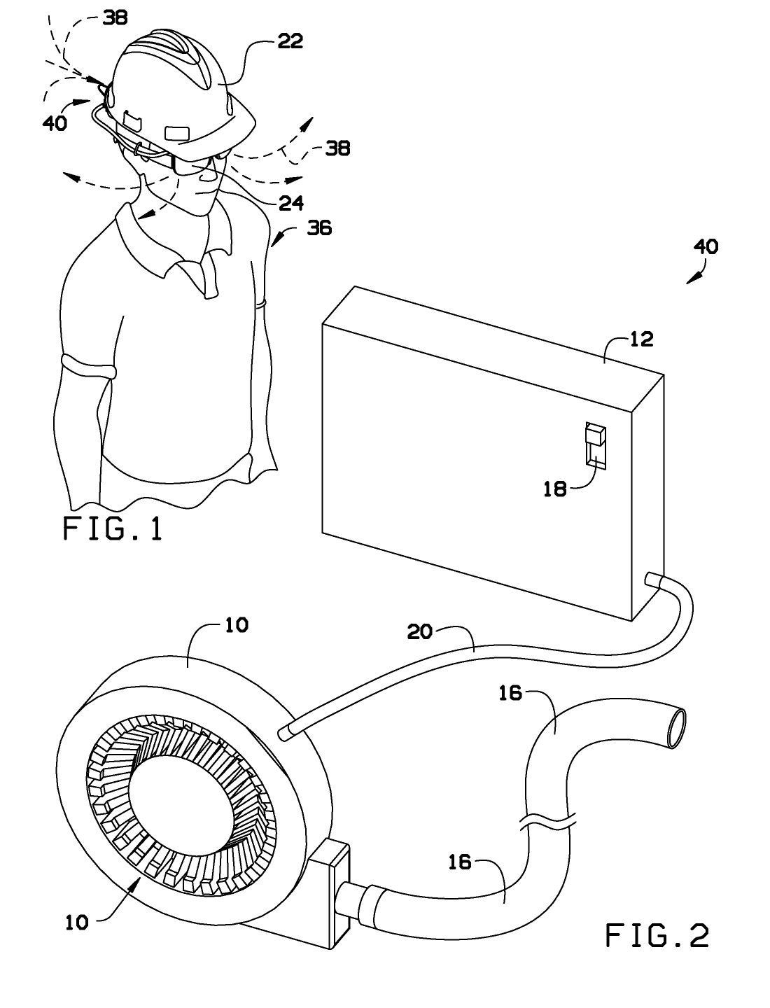

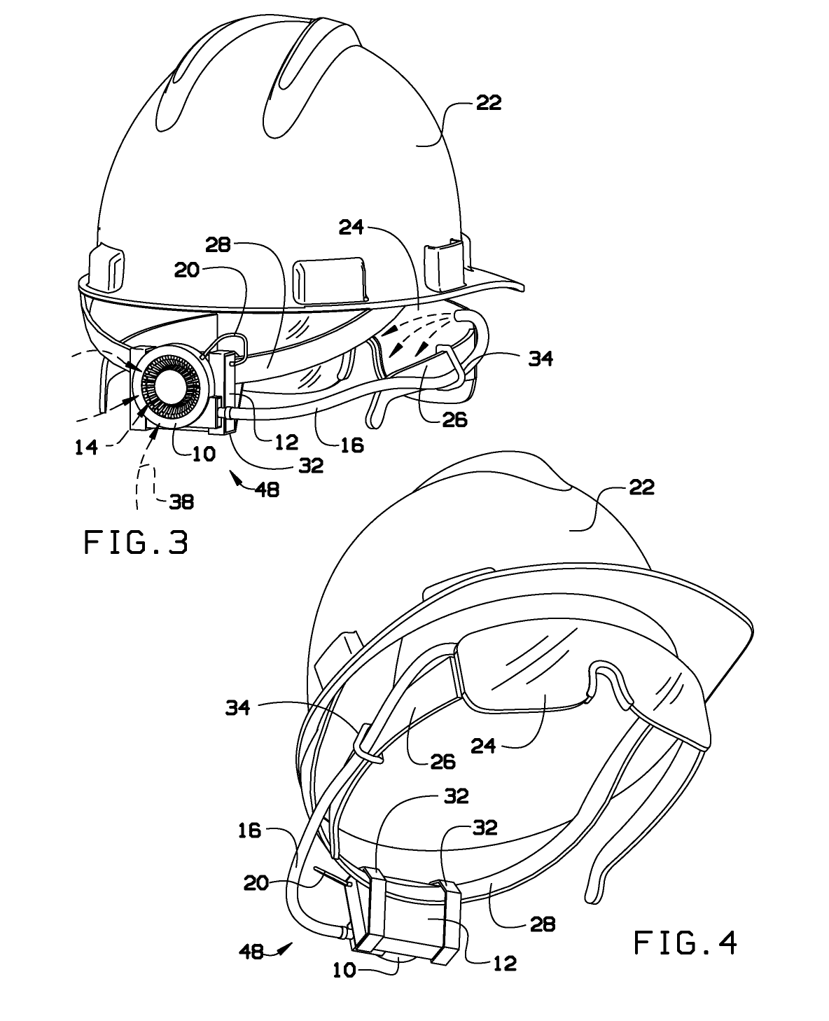

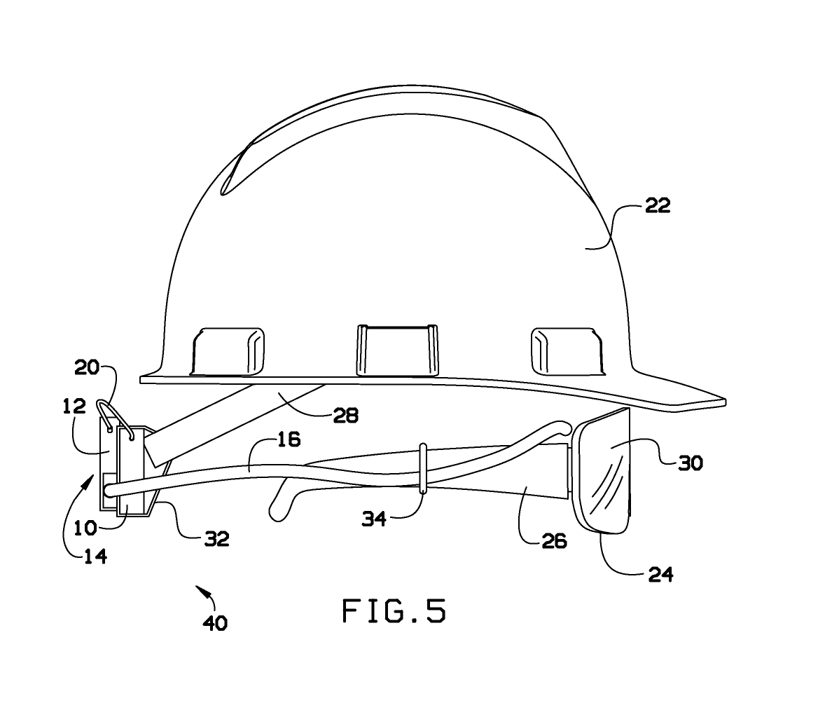

Referring now to FIGS. 1 through 5, a helmet or mask head unit 22 (hereinafter referred to as helmet 22) can include an attachment point 28 for securing a fan unit 10 and a battery unit 12 thereto. While the Figures show the attachment point 28 as a strap attached to the helmet 22, various other mechanisms can be used to attach the fan unit 10 and the battery unit 12 to the helmet 22. In some embodiments, attachment strapping 32 can be used to attach the battery unit to the attachment point 28 The fan unit 10 can be powered by the battery unit 12. In some embodiments, a power wire 20 can carry power from the battery unit 12 to the fan unit 10. The battery unit 12 can contain one or more batteries (not shown). The batteries may be rechargeable batteries or replaceable batteries. A power switch 18 can be disposed on the battery unit 12 to control delivery of power from the battery unit 12 to the fan unit 10.

In some embodiments, as shown in the Figures, the fan unit 10 may be attached to the battery unit 12. In some embodiments, the fan unit 10 and the battery unit 12 may be integrated into a single unit. In this embodiment, the power wire 20 may not be needed. In some embodiments, the fan unit 10 can be located some distance from the battery unit 12. For example, the battery unit 12 may be mounted directly to the helmet 22 while the fan unit 10 could be mounted on the attachment point 28, for example. In other embodiments, the fan unit 10 can be attached to the battery unit 12 to form an assembly 40, 48.

The fan unit 10 can be designed to provide an air flow 38 to pass through an air intake 14 of the fan unit 10 and deliver that air flow 38 to a desired location. This location can be, for example, on the user's head or up into the helmet 22 to help keep the user 36 cool and comfortable.

In some embodiments, an air outlet hose 16 can take some or all of the air flow 38 output from the fan unit 10 and direct that air flow 38 to a desired location. For example, the air outlet hose 16 can attach to an eyewear arm 26 of a user's eyewear 24 with attachment strapping 34 to direct the air flow 38 to the inside of the lens 30 of the eyewear 24. This can help remove particles from the inside of the eyewear 24 and also remove and prevent fogging of the lens 30 of the eyewear 24.

While the Figures show a single air outlet hose 16 providing the airflow 38 onto the lens 30 of the eyewear 24, two air outlet hoses 16 can be used to provide airflow 38 onto each lens 30 of the eyewear 24. In this embodiment, each air outlet hose 16 could run along each arm 26 of the eyewear 24.

It should be understood, of course, that the foregoing relates to exemplary embodiments of the invention and that modifications may be made without departing from the spirit and scope of the invention as set forth in the following claims.

What is claimed is:

1. A safety device accessory comprising:
a fan unit operable to create an airflow;
a battery unit operable to power the fan unit; and
an attachment mechanism for removably attaching the fan unit and the battery unit to a user, wherein
the airflow is directed to achieve at least one of 1) a first airflow into a helmet or mask of a user to provide user comfort, or 2) a second airflow onto a lens of eyewear of the user to prevent fogging thereof.

2. The safety device accessory of claim 1, further comprising a power wire electrically connecting the battery unit to the fan unit.

3. The safety device accessory of claim 1, wherein the fan unit and the battery unit are combined into a single assembly.

4. The safety device accessory of claim 1, further comprising an air outlet hose for directing the second airflow onto the lens of the eyewear of the user.

5. The safety device accessory of claim 4, further comprising at least one eyewear attachment strapping for securing the air outlet hose to an arm of the eyewear.

6. The safety device accessory of claim 1, further comprising at least one attachment strapping for securing the battery unit to an attachment point of the helmet or mask worn by the user.

7. The safety device accessory of claim 1, further comprising a power switch for controlling power delivery from the battery unit to the fan unit.

8. A safety device accessory comprising:
a fan unit operable to create an airflow;
a battery unit operable to power the fan unit; and
an attachment point for removably attaching the fan unit and the battery unit to a helmet or mask work by a user, wherein
the airflow is directed to achieve at least one of 1) a first airflow into the helmet or mask of a user to provide user comfort, or 2) a second airflow, delivered via an air outlet hose, onto a lens of eyewear of the user to prevent fogging thereof.

9. The safety device accessory of claim 8, further comprising at least one eyewear attachment strapping for securing the air outlet hose to an arm of the eyewear.

10. The safety device accessory of claim 8, further comprising at least one attachment strapping for securing the battery unit to an attachment point of the helmet or mask worn by the user.

11. The safety device accessory of claim 8, further comprising a power switch for controlling power delivery from the battery unit to the fan unit.

12. The safety device accessory of claim 8, wherein the fan unit and the battery unit are combined into a single assembly.
